(19) United States
(12) Patent Application Publication
(10) Pub. No.: US 2024/0180489 A1
(43) Pub. Date: Jun. 6, 2024

(54) WEARABLE SENSOR DEVICE FOR CONTACTING SKIN OF A PERSON

(71) Applicant: Agis Automatisering B.V., Harmelen (NL)

(72) Inventors: Joris Christian Beudel, Harmelen (NL); Gerard Marie Griffioen, Harmelen (NL); Robertus Gerardus Vanoudenallen, Harmelen (NL)

(21) Appl. No.: 18/552,062

(22) PCT Filed: Mar. 23, 2022
(86) PCT No.: PCT/NL2022/050157

§ 371 Date: Sep. 22, 2023

(30) Foreign Application Priority Data
Mar. 23, 2021 (NL) 2027818

(51) Int. Cl. A61B 5/00

(52) U.S. Cl. CPC A61B 5/6815; A61B 5/0059; A61B 5/6838; A61B 2562/164; A61B 2562/166

(57) ABSTRACT

A wearable sensor assembly for contacting skin, the sensor assembly comprises a hollow, flexibly deformable protrusion, a sensor module, and a sensor device fixating structure. The protrusion is preferably a cushion- or a balloon-shaped protrusion. The protrusion comprises a base and an apex, preferably a substantially flat apex. At least part of the apex forms a contact area for contacting the skin. The sensor module is mounted on or inside the protrusion, preferably on or against the contact area. The sensor device fixating structure is configured to press the contact area with a substantially constant pressure against the skin. When the contact area is pressed against the skin, an increase, respectively decrease of a force exerted on the sensor device deforms, e.g. deflects and/or compresses, the protrusion such that the contact area that is in contact with the skin increases, respectively decreases, while the pressure stays substantially the same.

### TECHNICAL FIELD

The invention relates to a wearable sensor device, and, in particular, though not exclusively, to a wearable sensor device for contacting skin of a person and a flexible sensor housing for use in such wearable sensor device.

### BACKGROUND

Currently in hospitals multiple vital signs of patients in high-care wards are being monitored continuously twenty-four hours a day. In contrast, when a patient is in a low-care hospital ward or at home, only selected vital signs such as pulse, oxygen saturation (SpO₂), photoplethysmography (PPG) measurements and temperature are monitored, and this is done manually at six to eight hours time intervals using spot measurements (usually referred to as a spot check). Typically, the measurements are manually entered into a (electronic) patient dossier. Patient safety can be improved, while working more efficiently at the same time when an automated trend of multiple vital signs could be measured.

For continuous monitoring of vital signs small wearable sensor devices are convenient, because devices do not restrict the mobility and movements of the patients. Typically, such devices include a sensor, e.g. an optical sensor, which has a contact area that is in contact with the skin of a patient. For example, the optical sensor may be configured to measure the optical response of the blood by exposing the skin to radiation of certain wavelengths. Such techniques are for example well known in the field of pulse oximetry. Typically, the sensor is held in place using a fixation structure, which may have a different design depending on the application, e.g. an adhesive structure or a clamping structure which fixates the contact area of the sensor to the skin of the patient.

WO2018/060291 describes an example of an ear sensor for measuring the vital signs based on an optical sensor. The ear sensor comprising an ear clamp (connected to or formed as part of the housing) configured to clamp the device to the user's ear. The sensor housing is in the form of a protrusion in the form of an anvil comprising a flexible connection that connects the protrusion to the housing. When worn, the clamping action of the ear clamp will bend the flexible connection so that the contact area of the sensor is pressed to the user's skin, regardless of the orientation between the contact surface and the skin. Similarly, US2013/0085356 describes examples of sensors with a pressure applicator in the form of a spring structure. The sensors include a sensor body, e.g. in the form of an adhesive plaster, comprising a substrate wherein the spring is positioned between the back of the sensor and the substrate to facilitate pushing of the optical element against the skin of the patient. WO2016/097271 describes a physiological parameter sensor device that can be worn around a finger. The device includes an actuator for controllably pressing an optical sensor with a predetermined force against the skin. The actuator can be controlled to provide a sensor contract pressure which allows for a maximized sensor signal.

These devices are typically based on non-invasive optical sensors which make theme very sensitive to motion. Despite the fixation structure, motion in the plane parallel to the contact area may cause changes in the reflection and/or transmission of the light. Similarly, motion in the plane perpendicular to the contact area may cause time-dependent pressure onto contact area causing changes in the reflection and/or transmission of the light and influencing the blood flow in the tissue directly under the contact area.

Motion may also cause an air gap between part of the contact area of the sensor and the skin. The motion may cause motion-induced noise in the sensor signal, causing irreproducibility in the measuring signal and inaccuracies in the vital sign parameters, including for example the blood perfusion level in the tissue based on PPG measurements and/or the blood oxygen saturation of hemoglobine in arterial blood (the SpO₂ level) in the artery blood, derived from the measured signal. Additionally, when using such sensors for long-term low care monitoring applications, long-term exposure to a force against skin will cause localized damage to the skin, also referred to pressure sores. The larger the pressure applied to the skin, the shorter the time before the skin gets irritated due to the pressure the sensor applies to the skin.

Although the above-mentioned prior art sensor structures include measures to fixate the contact area of the sensor to the skin of a user, it is very difficult to eliminate or at least decrease effects caused by motion while at the same time control the pressure that is needed for pressing the sensor area against the skin of the patient so that at the one hand a sufficiently reliable signal can be detected while at the other hand the sensor can be worn for a sufficiently long period so that it can be used as a monitoring device. In particular, for accurate measurement of PPG or SpO₂ levels it is desired that the sensor is in contact with the skin to the tissue with a predetermined constant contact pressure or with a contact pressure that is within a certain allowable range of contact pressures.

Hence, from the above it follows that there is a need in the art for an improved wearable sensor assembly that is configured to be in contact with the skin of a patient under all circumstances without influencing the blood flow and without the risk of the formation of skin irritation or even decubitus.

### SUMMARY

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a microprocessor of a computer. Furthermore, aspects of the present invention may take the form of a computer program product embodied in one or more computer readable medium(s) having computer readable program code embodied, e.g., stored, thereon.

It is an objective of the invention to reduce or eliminate at least one of the drawbacks known in the prior art.

In a first aspect, the invention relates to a wearable sensor device for contacting skin, comprising a flexibly deformable protrusion, preferably a hollow cushion- or a balloon-shaped protrusion. The protrusion comprises a base and an apex, preferably a substantially flat apex, and at least part of the apex forms a contact area for contacting the skin. The wearable sensor device further comprises a sensor module mounted on or inside the protrusion. Preferably, the sensor module is mounted on or against the contact area. The wearable sensor device further comprises a sensor device fixating structure configured to press the contact area with a substantially constant pressure against the skin, wherein, when the contact area is pressed against the skin, an increase, respectively decrease of a force exerted on the sensor device deforms, e.g. deflects and/or compresses, the protrusion such that the contact area that is in contact with the skin increases, respectively decreases, while the pressure stays substantially the same.

Thus, the wearable sensor assembly may fixate a contact surface of the protruding element comprising a sensor module, a sensor head, against the skin of the user. Then, when a force is exerted on the protruding element due to e.g. a movement of the user, the protruding element will deflect and/or compress. This way, contact can maintained between the contact surface of the protrusion without substantially increasing the contact pressure between the sensor head and skin so that a detrimental increase of the pressure/stress exerted on the skin can be avoided. The sensor assembly may provide a high-quality sensor signal while only exerting a minimal pressure on the skin. This way, the sensor can be worn for a longer period of time while minimizing the chance of skin irritation. Advantageously, the substantially constant pressure is maintained in a passive way. Thus, there is no need for an active feedback system comprising, e.g., a pressure sensor and an actuator. As a result, the dimensions of the sensor device can remain small and lightweight, increasing possibilities for wear (e.g., on an ear) and increasing comfort, while at the same time reducing production cost and power demands (leading to, e.g., an increased battery life).

In an embodiment, in an embodiment, the pressure may be smaller than 8 kPa, preferably smaller than 6 kPa, more preferably smaller than 4 kPa, even more preferably smaller than 3 kPa.

In an embodiment, the sensor module may be a reflective type sensor module including at least one light emitting element (e.g. a LED) and a light detecting element. In an embodiment, the light emitting and light detecting element may be arranged next to each other fixated against an inner side of the hollow deformable protrusion.

In an embodiment, the lateral distance between the (middle) of first sensor element and the second sensor element may be selected between 2 and 20 mm, preferably between 4 and 16 mm.

In an embodiment, the base may be configured as a flexible joint region connecting the protruding element to the fastening structure, preferably the flexible joint region providing flexible movement of the protrusion in rotational, translational and/or angular directions relative to the base part.

In an embodiment, at least part of the sensor module may be fixated inside the protrusion against a sheet material forming the contact area of the protrusion.

In an embodiment, the hollow flexibly deformable protrusion may be made of an elastomeric sheet material, preferably a silicone material, preferably the thickness of the sheet material forming the sidewalls and/or the contact area is selected between 0.2 and 4 mm, preferably between 0.4 and 2 mm.

In an embodiment, at least part of the contact area is made of a flexible opaque material, the contact area further comprising one or more transparent windows of a flexible optically transparent material, preferably the sensor module being an optical sensor module comprising one or more optical sensor elements, the optical sensor module being mounted against the backside side of the contact area in alignment with the one or more transparent windows.

In an embodiment, the surface the contact area may comprise one or more recesses at the position of the one or more transparent windows, the one or more recesses being configured to house at least part of the one or more optical sensor elements respectively.

In an embodiment, the sensor module may comprise at least a light detecting element and a light emitting element, optionally a light blocking member being positioned between light detecting and the light emitting element for optically isolating the light detecting element from light that arrives at the light detecting element other than via one of the optical windows.

In an embodiment, the protruding element may comprise a cavity or a chamber formed by the apex and one or more side walls connecting the apex to the base, preferably the apex and the one or more side walls being of a flexible sheet material, preferably a silicone or a rubber material.

In an embodiment, at least part of the one or more elastic walls may have a convex shape so that when the protruding element is deflected and/or compressed, the one or more side walls will deflect outwardly.

In an embodiment, the cross-sectional dimensions of the protruding element at the joint region, preferably a circumference and/or a width at the joint region, may be smaller than the cross-sectional dimensions of the protruding element at a height of the side walls.

In an embodiment, the sensor device further may comprise a housing comprising electronics for processing sensor signals of the sensor module, one or more strips of a flexible PCB electrically connecting the sensor module to the electronics, preferably the one or more strips of the flexible PCB comprising a plurality of thin-film conductors on a thin dielectric film.

In an embodiment, the sensor module may include a first sensor element mounted against a first part of an inner side of the material forming contact area and a second sensor element mounted against a second part of the material forming the inner side of the contact area so that, when the outer surface of the contact area is pressed against the skin, the first sensor element can have an orientation that that is different from the orientation of the second sensor element.

In an embodiment, the sensor module may comprise an elongated rigid PCB board having a longitudinal axis and a transversal axis, the rigid PCB comprising two or more sensors elements mounted thereon and at least two flexible PCB strips, each flexible PCB strip being connected to a long side of the rigid PCB board so that the rigid PCB board can rotate about its longitudinal axis and its transversal axis.

In an embodiment, the sensor device fixating structure may be configured to fixate, preferably mechanically fixate, the contact area against the skin of the backside of the ear, preferably the module fixating structure including a flexible clamping structure for clamping the sensor module around the ear to that the contact area is pressed against the skin of the backside of the ear.

In an embodiment, the wearable sensor device may be configured as an ear sensor, the sensor device fixating structure including an ear hook of an elastic material configured to press the contact area against skin of the backside of the ear, a first end of the ear hook being sphere shaped contact structure, the sphere shaped contact structure a spherical element and a balloon-structure of a flexible sheet material formed around the spherical element.

In an embodiment, the wearable sensor device may be configured as wrist-worn sensor device or a plaster.

In an embodiment, the sensor may comprise a protective casing comprising a top surface, side walls and a bottom surface; the bottom surface including a peripheral part, the flexibly deformable protruding element being flexibly connected to the peripheral part, the peripheral part including a surface for positioning the sensor device on a part of the wrist.

In a further embodiment, the sensor device fixating structure may be connected to the side walls and including an wristband to hold the peripheral part against the skin, wherein the contact area of the protruding element extends beyond the surface of the peripheral part, so that when sensor device is fixated against the skin, the contact area will be pressed against the skin with a predetermined pressure.

In a further aspect, the invention may relate to a wearable sensor device for contacting skin comprising: a flexibly deformable protrusion, preferably a cushion- or balloon-shaped protrusion, comprising a base and an apex, at least part of the apex forming a contact area for contacting the skin; a sensor module mounted on or inside the protrusion; and, a fixating structure, wherein the sensor module includes a first sensor element flexible connected to a second sensor element so that, when the contact area is pressed by the fixating structure against a curved part of the skin, the first sensor element has a first orientation and the second sensor element has a second orientation that is different from the first orientation. The sensor elements can be orientated independently form each other so that the sensor elements are always in contact with the skin. Although the absolute path length may differ per person, the relative ratio between the pathlength of the two different wavelengths will be the same so that the path lengths can be removed from the computation. This way, only one calibration curve is needed. A further advantage is that the sensor elements, the LEDs and the photosensor may be spaced further apart as they will follow the curved surface of the skin. This will increase the perfusion underneath the sensor and hence increase the quality and reliability of the sensor signal.

In an embodiment, the first sensor element, preferably one or more light emitting elements, e.g. LEDs, may be mounted against a first part of an inner side of the contact surface and the second sensor element, preferably a light receiving element, may be mounted against a second part of the inner side of the contact surface.

In an embodiment, the sensor module may be a reflective type sensor module.

In an embodiment, the lateral distance between the (middle) of first sensor element and the second sensor element may be selected between 2 and 20 mm, preferably between 4 and 16 mm.

In an embodiment, the sensor device may further comprises a housing comprising electronics for processing sensor signals of the sensor module, at least a first and second flexible PCB electrically connecting the first and second sensor elements to the electronics respectively, preferably the first and second flexible PCB comprising a plurality of thin-film conductors on a thin dielectric film.

In an embodiment, the fixating structure may be configured to press the contact area with a substantially constant pressure against the skin, wherein, when the contact area is pressed against the skin, a force exerted on the sensor device will deform, e.g. deflect and/or compress, the protrusion such that the contact area that is in contact with the skin increases, while the pressure stays substantially the same.

In an embodiment, in an embodiment, the pressure may be smaller than 8 kPa, preferably smaller than 6 kPa, more preferably smaller than 4 kPa, even more preferably smaller than 3 kPa.

In an embodiment, the base may configured as a flexible joint region connecting the protruding element to the fastening structure, preferably the flexible joint region providing flexible movement of the protrusion in rotational, translational and/or angular directions relative to the base part.

In an embodiment, the flexibly deformable protrusion may be hollow and made of an elastomeric sheet material, preferably a silicone material, preferably the thickness of the sheet material forming the sidewalls and/or the contact area is selected between 0.2 and 4 mm, preferably between 0.4 and 2 mm.

In an embodiment, at least part of the sensor module may be fixated inside the protrusion against a sheet material forming the contact area of the protrusion.

In an embodiment, at least part of the contact area may be made of a flexible opaque material, the contact area further comprising a first and second transparent window of a flexible optically transparent material, the first and second sensor element being mounted against the backside side of the contact area in alignment with the first and second transparent windows respectively.

In an embodiment, the surface of the contact area may comprise at least first and second recesses at the position of the first and second window respectively, the first and second recesses being configured to house at least part of the first and second sensor elements respectively.

In an embodiment, a light blocking member may be positioned between first and second sensor element for optically isolating the first sensor element from light that arrives other than via the first optical window.

In an embodiment, the protruding element may comprise a cavity or a chamber formed by the apex and one or more side walls connecting the apex to the base, preferably the apex and the one or more side walls being of a flexible sheet material, preferably a silicone or a rubber material.

In an embodiment, at least part of the one or more elastic walls have a convex shape so that when the protruding element is deflected and/or compressed, the one or more side walls will deflect outwardly.

In an embodiment, the cross-sectional dimensions of the protruding element at the joint region, preferably a circumference and/or a width at the joint region, may be smaller than the cross-sectional dimensions of the protruding element at a height of the side walls.

In an embodiment, the sensor device fixating structure may be configured to fixate, preferably mechanically fixate, the contact area against the skin of the backside of the ear, preferably the module fixating structure including a flexible clamping structure for clamping the sensor module around the ear to that the contact area is pressed against the skin of the backside of the ear.

In an embodiment, the wearable sensor device may be configured as an ear sensor, the sensor device fixating structure including an ear hook of an elastic material configured to press the contact area against skin of the backside of the ear, a first end of the ear hook being configured as a sphere-shaped contact structure, the sphere-shaped contact structure comprising a spherical element and a balloon-structure of a flexible sheet material formed around the spherical element.

The wearable sensor device may also be configured as a wrist sensor or a plaster in accordance with any of the embodiments described in this application.

In yet another aspect, the invention may relate to a wearable ear sensor device for contacting skin of the backside of the ear, wherein the sensor device may comprise: a flexibly deformable protrusion, preferably a cushion- or balloon-shaped protrusion, comprising a base and an apex, at least part of the apex being configured as a contact surface for contacting the skin; a sensor module mounted on or inside the protrusion; and, an ear hook of an elastic material configured to press the contact surface of the deformable protrusion against the skin, a first end of the ear hook comprising a balloon-shaped contact structure, the balloon-shaped contact structure comprising a spherical element connected to the ear hook and a balloon-structure of a flexible sheet material formed around the spherical element, wherein the radius of the balloon structure is larger than the radius of the spherical element so that an airtight volume is formed.

The airtight space may be filled with air or a gas wherein when the sensor is not in use the pressure within the airtight space is approximately equal to the pressure outside the space. This way, the silicone balloon will have a compressible sphere-shaped contact surface. This way, when the ear sensor is worn around, the contact pressure between the balloon-shaped contact structure can be kept below 7 kPa and, preferably at an (average) contact pressure between 2-3 kPa on average. This way, skin irritation or even skin damage due to as long-term exposure to a force against skin can be avoided.

The wearable ear sensor device the balloon-shaped contact structure may further include any of the features described with reference to the embodiments above.

The invention will be further illustrated with reference to the attached drawings, which schematically will show embodiments according to the invention. It will be understood that the invention is not in any way restricted to these specific embodiments.

### BRIEF DESCRIPTION OF THE DRAWINGS

FIG. 1A-1C depict a wearable sensor assembly according to an embodiment of the invention;

FIGS. 2A and 2B depict graphs regarding the sensor signal as a function of the contact pressure and the contact pressure as a function of the load time;

FIG. 3 depicts a cross-section of the hollow, flexibly deformable protruding element housing a sensor module according an embodiment of the invention;

FIGS. 4A and 4B depict cross-sectional view of the flexibly deformable protruding element according to an embodiment of the invention;

FIGS. 5A and 5B depict cross-sectional view of a flexibly deformable sensor housing according to an embodiment of the invention;

FIGS. 6A and 6B depict a cross-sectional view of flexibly deformable according to an embodiment of the invention.

FIG. 7 depicts a sensor module connected to flexible printed circuit board wiring according to an embodiment of the invention;

FIG. 8 depicts a sensor assembly comprising a sensor module connected to flexible printed circuit board wiring according to an embodiment of the invention;

FIG. 9A-9C illustrate a flexible printed circuit board structure connected to a sensor module and a flexible protruding element for a sensor assembly according to another embodiment of the invention;

FIG. 10A-10C illustrates two sensor elements having different orientations when the flexibly deformable sensor housing is pressed against an irregular surface;

FIGS. 11A and 11B illustrate an example of ear hook for an ear worn sensor device according to an embodiment of the invention;

FIG. 12 depicts the relation between the opening distance between the two contact surfaces of the ear sensor and the spring constant of the ear hook;

FIGS. 13A and 13B illustrate the elastic properties of the balloon-shaped contact structure of the ear hook according to an embodiment of the invention;

FIGS. 14A and 14B depict a wrist-worn sensor device according to an embodiment of the invention.

FIGS. 15A and 15B depict a wrist-worn sensor device according to an embodiment of the invention.

FIGS. 16A and 16B depict a sensor device according to an embodiment of the invention that is configured as a plaster or a patch

FIG. 16 depicts a schematic of functional components a wearable sensor assembly according to the embodiments in this application could comprise;

FIG. 17 depicts a schematic of a vital sign monitoring system according to an embodiment of the invention;

### DETAILED DESCRIPTION

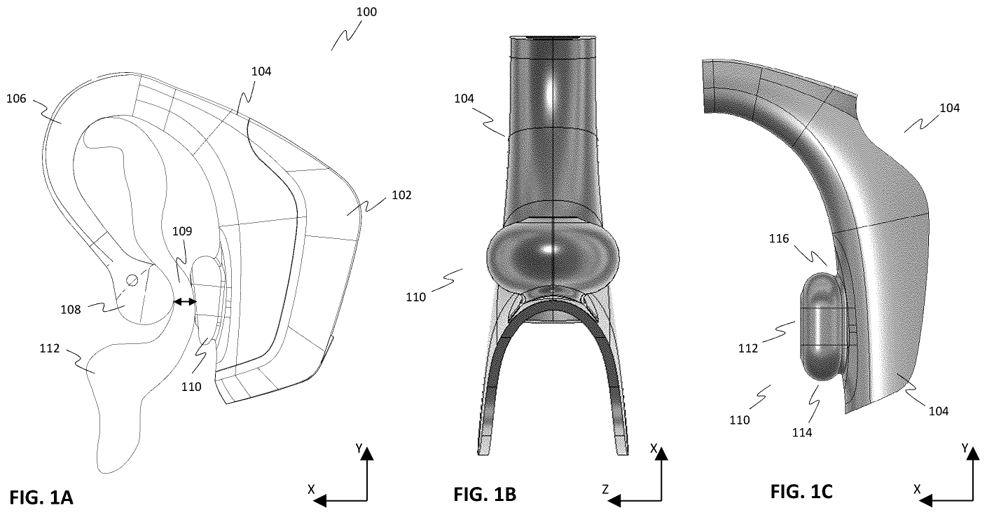

The embodiments in this disclosure describe wearable sensor devices for accurately monitoring vital sign parameters. The sensor devices are adapted to provide reliable data acquisition and determination of vital sign parameters, while allowing comfortable wear of the sensor for a prolonged time without skin irritation. FIG. 1A-1C depict a wearable sensor device according to an embodiment of the invention. In particular, FIG. 1A illustrates side-view of a sensor device 100 comprising an electronics compartment 102, fastening structure 106, 108 connected to the electronics compartment and a flexible sensor housing structure 104 which may be attached to or form part of the electronics compartment. The flexible sensor housing structure may comprise a protruding element, a protrusion, comprising a sensor module (e.g. an opto-electronic sensor module) that needs to be in close contact with skin tissue under all circumstances.

The protrusion may form a deformable sensor head 110 in the shape of a cushion or a flattened balloon. The protruding element may be flexibly connected to the electronics housing that houses the electronics for processing sensor signals and for wirelessly transmitting processed sensor signals to a base station. As shown in FIG. 1A, in an embodiment, the wearable sensor device may be an ear-worn sensor device fixated around part of the ear 112 of the wearer (for clarity reasons the figure only illustrates a cross-sectional part of the ear). The ear-worn sensor device may include an elastomeric arm 106, forming ear hook, that is shaped to be worn around the ear. The elastomeric arm may include a first end connected to the electronics housing 102 and the protruding element 110 and a second end that is shaped as a sphere 108.

When worn around the ear, the spherical end is pressed against the inner side of the ear, while the protruding element comprising the sensor module is pressed against the backside of the ear. The elastomeric arm functions as spring allowing a part of the ear to be clamped between the two ends. In particular, when the ear hook is worn around the ear 112, a contact area of the cushion-shaped protruding element may be pressed against the back side of the ear while the sphere-shaped end is pressed against the inner side of the ear in a direction that is substantially opposite to the direction the protruding elements pushed against the back side of the ear. The clamping action of the elastomeric arm will keep the sensor device fixated.

The thickness of a human ear (inner ear—backside ear) may vary between 2 and 5 mm, typically between 2.5 and 4.5 mm. Further, the curvature of the backside and the inner ear may vary substantially. Additionally, the surface of the backside of the ear may have an irregular surface. Hence, designing ear hook that keeps the sensor module in contact with skin tissue under all circumstances, without damaging the skin is a non-trivial problem. Although the embodiment shown in FIG. 1A illustrates an ear worn sensor device, the invention is not limited to ear sensors but can also be implemented as other sensor types such as a sensor device worn around the arm of a sensor device that can be applied to the body as a plaster. Examples of such sensor devices are described hereunder in more detail. The sensor module may be an optical sensor module configured to optically measure vital sign parameters, such as temperature, the heart rate and/or SpO₂ levels in the blood of the patient wearing the sensor assembly.

It is known in the art that motion will cause motion-induced noise in the sensor signal. For example, in case of a reflective type optical sensor, movements in the plane parallel to the contact area may cause changes in the reflection and/or transmission of the light. Similarly, motion in the plane perpendicular to the contact area may cause time-dependent pressure onto contact area influencing the blood flow in the tissue directly underneath the contact area. Moreover, motion may also cause an air gap between part of the contact area of the sensor and the skin. Such air gap will significantly influence reflection and/or transmission of the optical signal that is measured by the sensor module. Motion-induced noise in the sensor signal may cause irreproducibility in the measured signal and inaccuracies in the vital sign parameters, such as the blood perfusion level in the tissue based on PPG measurements and/or the blood oxygen saturation of hemoglobine in arterial blood (the SpO₂ level) in the artery blood.

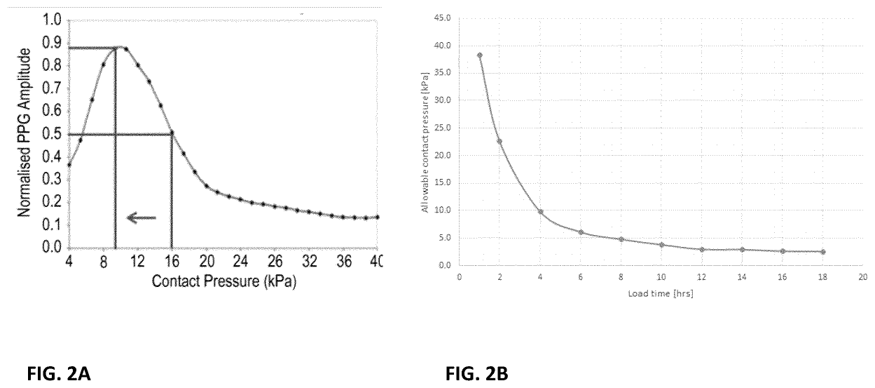

It is known in the art that the signal quality of the optical sensor is dependent on the contact pressure between the sensor and the skin. For example, FIG. 2A depicts a figure taken from WO2016/097271 indicating that a contact pressure of about 10 kPa provides an optimal signal quality for PPG measurements. However, it is known that such contact pressure only allows a limited time for wearing the sensor assembly without skin irritation as long-term exposure to a force against skin will cause localized damage to the skin, also referred to pressure sores. The larger the pressure applied to the skin, the shorter the time before the skin gets irritated due to the pressure the sensor applies to the skin. FIG. 2B for example depicts a graph of the allowable contact pressure in kPa as a function of the load time. This graph shows that a contact pressure of 10 kPa (optimal for data acquisition) only allows a load time of approximately 4 hours or less. A pressure of 10 kPa however will affect the blood perfusion in the veins. To avoid such effects the pressure should at least be smaller than 6-7 kPa. This graph illustrates that for long time loads, e.g. 8 hours, the contact pressure preferably should be smaller than 5 kPa which significantly reduces the signal quality. Further, for a sensor device that is worn for several days at the same position the contact pressure should be not higher than 2 to 3 kPa.

To address these problems, the sensor assemblies described with reference to the embodiments in this application include various measures for improving the signal quality and/or long-term wearability of sensor device for difference ear geometries. FIGS. 1B and 1C depict part of the flexible sensor housing structure as shown in FIG. 1A in more detail. As shown in the figure, the sensor housing structure may be designed to minimize the risk of sub-optimal contact between optical sensor and the skin. To that end, in an embodiment, the sensor housing may be a molded silicone structure including a sensor head connected at its base to a fastening structure, e.g. a silicone sheet that is shaped to fit around (at least part of) the electronics housing and the ear hook. The sensor head comprising the sensor module may be shaped as a protrusion 110 that exhibits spring-like properties in different directions. The hollow shape may act as a preloaded spring. Thus, when attaching the sensor device to the ear using the ear hook as shown in FIG. 1A, the flexible sensor head will be pressed against the skin of a user with a pressure that is within a certain range regardless of the orientation of the contact surface and regardless of forces exerted onto the sensor device due to e.g. movements of the user.

To achieve these effects, the sensor housing may include a hollow flexibly deformable protruding element 110, for example a cushion-shaped protruding element, having a base 116 and an apex 112 and convex-shaped side walls 114 connecting the apex with the base. The circumference of the protruding element at the base may be smaller than its circumference at a certain position between the base and the contact center. This way, the base of the protruding element may form a flexible joint of the protruding element. Further, at least part of the apex may be configured as a contact surface for contacting the skin. The protruding element may be hollow, e.g. have a cavity or chamber. Further, the hollow protruding element may be configured to house the sensor module wherein at least part of the sensor module may be fixated against the inner side of the contact surface. The outer edge of the contact surface may be have an elliptical form, including a longer axis and a shorter axis. This way, the contact surface allows an elongated sensor module comprising multiple sensor elements against the inner side of the sheet material forming the contact surface. The fastening structure of the sensor housing structure may be used to attach the sensor head to the electronics compartment.

The ear hook of the sensor device may define a fastening structure 106, 108 connected to the electronics compartment which is configured to fixate the sensor device to part of the body of the wearer, e.g. an ear, arm or other body part, so that the contact surface of the sensor head is always in contact with the skin. For example, in the figure, the fastening structure may be configured as an ear clamp so that the sensor assembly can be used as an ear sensor. The fastening structure will fixate the contact surface of the protruding element against the skin of the user. Then, when a force is exerted on the protruding element due to e.g. a movement of the user, the protruding element will deflect and/or compress, thus maintaining contact between the contact surface of the protrusion and the skin while preventing a detrimental increase of the pressure/stress exerted on the skin. This way, a high-quality sensor signal can be achieved while only exerting a minimal pressure on the skin. The sensor may be worn for a longer period of time, while minimizing the chance of skin irritation.

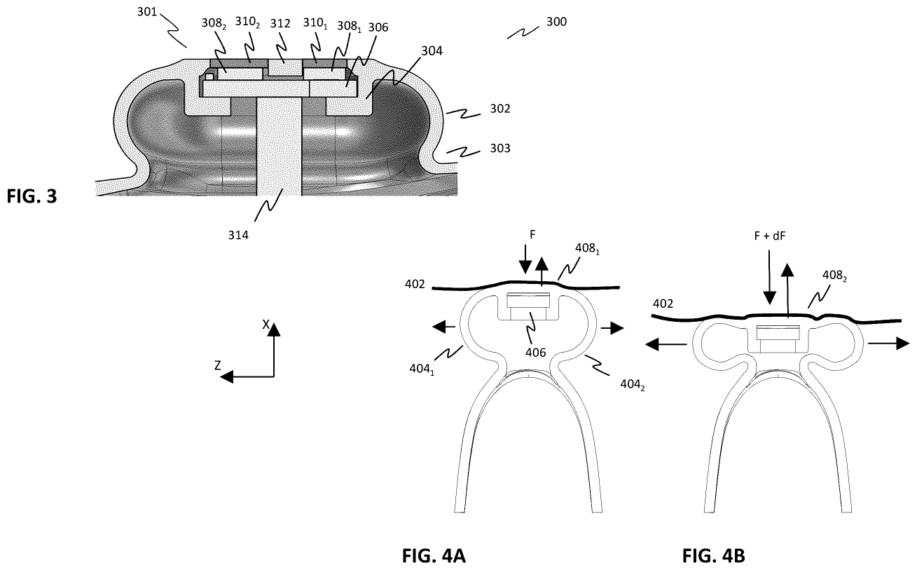

FIG. 3 depicts a cross-section of the hollow, flexibly deformable protruding element 300 in more detail. The deformable protruding element may be hollow, i.e. have a chamber or a cavity. The cavity may be formed by the flexible side walls 302 that connecting a contact surface 301 (the apex) via a base 303 to the electronics compartment (not shown). The contact surface and the one or more side walls may be made out of a flexible material such as a silicone or a rubber material that has a high friction coefficient with dry skin. In an embodiment, the deformable protruding element may be formed based on a molding technique such as an injection molding. The deformable protruding element (the “balloon”) may have a height (determined from the base) of between 3-12 mm, preferably between 4-10 mm. Further, the thickness of the flexible sheet material may be selected between 0.2-0.8 mm. In case of a protruding element having an elliptically shaped contact area, the protruding element may be between 6 and 16 mm along its long axis and between 4 and 12 mm along its short axis.

The protruding element may be configured to house a sensor module 306. In an embodiment, the sensor module may comprise a substrate, e.g. a rigid or a semi-rigid PCB, and one or more sensor devices mounted thereon. The sensor module may be fixated against the inner side of the contact surface of the protruding element using a fixating structure 304 which may mechanically fixate the sensor module. The fixating structure may form an integral part of the protruding element. For example, the fixating structure may include a recess around the inner side of the contact surface. The recess structure may have dimensions that match the sensor module so that the sensor module can mechanically clamped against the inner side of the contact surface.

The sensor module may include one or more sensor elements 308_{1,2}. In an embodiment, the senor elements may include a light emitting device such as a laser or the like and a light detecting device such as a photo detector. The fixating structure may fixate the optical sensor module such that the sensor elements are aligned with the optical transparent windows 310_{1,2} in the contact surface. At the position of the transparent windows the inner side of the contact surface may include recessions that may engage with the optical elements. Further, an optically isolating member 312 may be positioned between the optical transparent windows so that the sensor elements can only receive or emit light through the optical transparent windows. This way “optical leakage” of light of the light emitting device through the housing can be avoided. The optical windows in the sensor head be realized based on a two-step injection molding process wherein in a first step a first mold may be used to realize the opaque part of the silicone structure and in a second step a second mold may be used to realize the transparent windows.

In case of the ear-worn sensor as depicted in FIG. 1, the elastic properties of the sensor device may be controlled by the electric properties of the elastic arm and the elastic properties of the protruding element. The spring constants of the arm and the protrusion may be may carefully designed such that when places around the ear, the contact pressure with which the protrusion and the end of the elastic arm presses against the inner side and the back side of the ear respectively. For example, realize contact pressures within the range that allows long term wear of the sensor device, the spring constant of the protruding element may be selected between 5 and 100 mN/mm, preferably between 10 and 80 mN/mm, more preferably between 20 and 60 mN/mm.

Due to the low spring constant of the protrusion, it is very sensitive to any additional spring loading effects of for example electronic wiring connecting the sensor module that is fit to the electronics. To minimize such spring loading effects, a narrow, thin flexible printed circuit board 314 may be used to connect the sensor module to the electronics in the electronics compartment. Here, the width of the narrow flexible printed circuit board may be between 1 and 5 mm, preferably between 2-4 mm. The narrow thin flexible printed circuit board may comprise thin-film conductors on a flexible thin dielectric film, wherein at least part of the thin-film conductors connect the sensor module to the electronics in the electronics compartment. The geometry and the mechanical properties of the thin flexible printed circuit board will be discussed hereunder in greater detail.

FIGS. 4A and 4B depict cross-sectional view of the flexibly deformable protruding element according to an embodiment of the invention. In FIG. 4A, the protruding element may be pressed against the skin 402 of person with a light force F wherein the force is parallel to the x axis. When a small enough force is applied, the spring-like properties may cause a small compression of the protruding element until the applied force and the reactive spring force is in balance as shown in FIG. 4A. In this situation, a certain part of the contact area 408₁ may be in contact with the skin defining a certain contact pressure area, which should be large enough to keep the sensor in position. A movement of the user may cause an increase of the force dF applied to the flexibly deformable protruding element. In that case, the sensor head may be compressed in a direction towards the base (along the x-axis) and the convex-shaped elastic side wall may be deflected outwardly (along the z-axis) as shown in FIG. 4B. Thus, the increase of the force exerted on the sensor assembly may cause the protruding element to deflect and/or compress such that the contact surface area 408₂ that is in contact with the skin increases. The increase in the contact area may keep the contact pressure at the position where the protruding element is in contact with the skin within certain boundaries. This way contact between the protrusion/sensor(s) and the skin can be maintained while a detrimental increase of the pressure/stress exerted on the skin can be eliminated or at least minimized.

In an embodiment, the chamber or cavity of the protruding element may be substantially airtight or may have a small opening allowing gas, e.g. air, to go in or out depending on the pressures difference between the pressure inside the cavity and outside the cavity. In an embodiment, the cavity may be filled with a gas, e.g. air, at a pressure that is approximately equal to the pressure outside the cavity. This way, the protruding element may act as an air suspension which may contribute to the overall spring properties of the protruding element. In particular, it may contribute to the stability and/or wear conform of the sensor device. In an embodiment, the pressure in the cavity may be larger than the pressure outside the cavity. Combining a gas pressurized cavity with flexible wall, allows the protruding element to be pressed against skin at a constant pressure. In further embodiments, instead of a gas, the cavity may be filled with another compressible material such as a foam, such as a memory foam of the like.

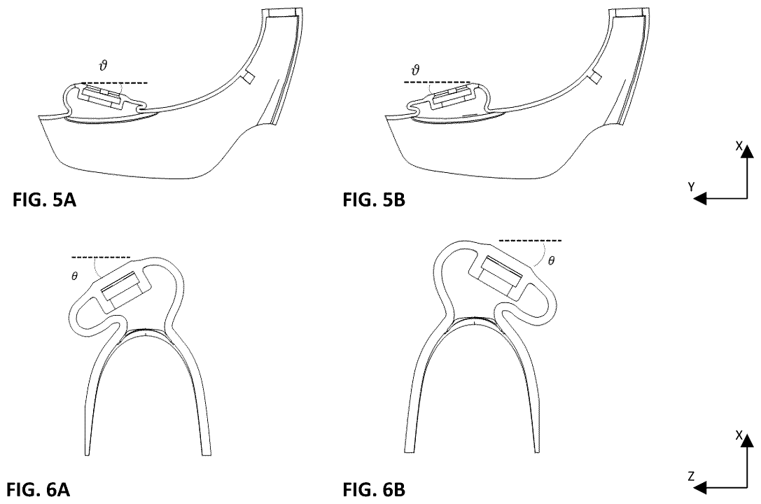

FIGS. 5 and 6 illustrate additional advantageous properties of the hollow, flexibly deformable protruding element according to an embodiment of the invention. Part of the base of the protruding element maybe configured as a flexible joint region connecting the protruding element to the fastening structure. The flexible joint region may be configured to allow flexible movements of the contact surface in rotational, translational and/or angular directions relative to the base part. The dimensions (for example the circumference) of the protruding element in the y-z plane at the joint region may be smaller than the dimensions of the protruding element in the y-z plane at the position where its circumference is maximal (e.g. at the height of the convex-shaped side walls). Additionally, the side walls at the joint region may be concave-shaped allowing the protruding element to contact the skin of the user under different angles as e.g. illustrated in FIGS. 5A and 5B by an angle ϑ representing a rotation around the z axis. Here, the protruding element may be configured to allow rotations up to +20 degrees relative to the rest position of the protrusion. Similarly, FIGS. 6A and 6B illustrate an angle θ representing a rotation around the y axis, which may allow rotations up to +40 degrees. The maximum angles will depend on the design (dimensions and shape) of the protruding element.

The sensor module in the protruding element needs to be electrically connected to the electronics that is housed in the electronics compartment of the sensor. As already mentioned before, simply connecting the sensor module with the electronics using conventional wiring would indivertibly affect the mechanical properties of the protruding element. Therefore, a thin flexible PCB structure is designed such that its influence on the spring properties of the protruding element are minimized.

FIG. 7 illustrates a flexible printed circuit board structure connected to a sensor module that can be used in sensor assemblies as described with reference to the various embodiments in this application. In particular, the figure illustrates a flexible printed circuit board structure, including two narrow flexible PCB strips 710_{1,2} connected to a sensor module 704. The two narrow flexible PCB strips are then joined into one PCB strip that is connected to electronics 703, e.g. an analog stage for processing signals originating from the sensor module.

In this embodiment, the sensor module may include a rigid or a semi-rigid substrate 705 of an elongated shape (having e.g. a longitudinal axis in the y-axis in this example) so (at least) two sensor elements 706, 708 can be arranged onto the substrate, next to each other in the direction of the longitudinal axis. The PCB may be connected to a thin flexible PCB structure that so that, when mounted inside the protruding element, the electrical connection provided by the flexible PCB board only minimally influences the mechanical characteristics of the protruding element. To that end, a flexible PCT strip 702 connected to the electronics may be spitted in two narrow flexible PCB strips 710_{1,2} so that electrical connections to both sides (the long sides) of the sensor module can be realized.

As shown in the figure, for electrical connection of the sensor module to the electronics one or more (in this example two) flexible PCB strips may be connected to the long sides of the elongated rigid or semi-rigid PCB such that the flat surface of the flexible PCB strips are parallel to the surface of the rigid or semi-rigid PCB. In an embodiment, the width of the flexible PCB strips may be selected between 3-8 mm. Further, each flexible PCB strip may include a connection part 712_{1,2} connecting the flexible PCB strip to the rigid PCB. Both strips may be connected to both sides of the rigid PCB at approximately the center of the long side. Connecting the PCB strips this way at both sides will balance minor forces that the PCB trips may exert onto the sensor module thus minimizing effects of the PCB strips connected to the sensor module as much as possible. As shown in the figure, in an embodiment, the connection part extends may extend from the edge of the rigid PCB in a direction that is substantially perpendicular (e.g. the y direction) to the longitudinal axis of the rigid PCB (e.g. the x direction).

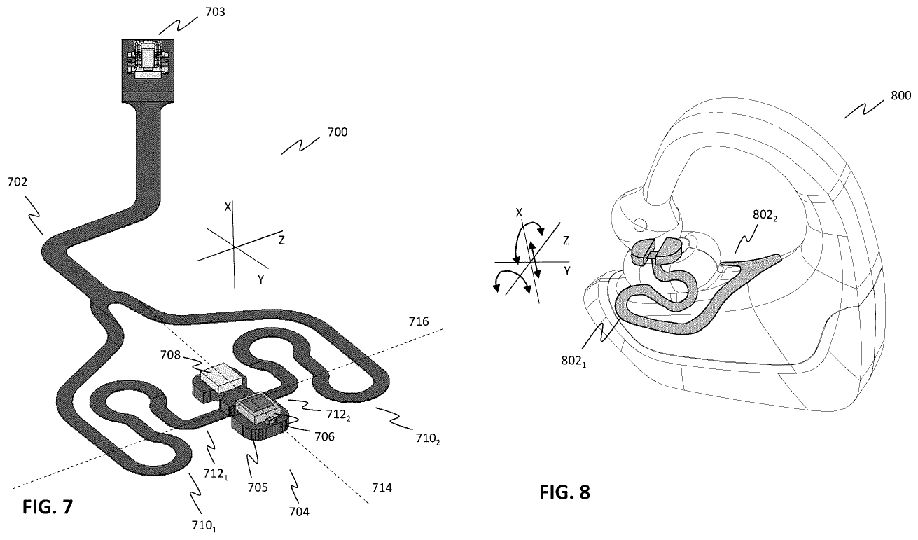

Further, each flexible PCB strip further includes a meandering part 710_{1,2} that allows the flexible PCB to be folded into the protruding element as depicted in FIG. 8. The PCB strips have different flexibility properties in plane and out of plane. The meandering parts are introduced to tackle this problem. The meandering parts provide sufficient length to the PCB strips to allow the strips to be slightly bent and/or twisted into a 3D wiring arrangement that fits the limited space of the protruding element and the sensor assembly.

In a further embodiment, instead of one or more flexible PCB strips, one or more strips of a flexible and stretchable printed circuit board material may be used to electrically connect the sensor module with the electronics. Such stretchable PCB may include thermoplastic polyurethane substrate on which meandering conductive strips or conductive polymer strips are provided to enable stretchability of the PCB.

As shown in this picture, when mounted in the protruding element, the two flexible meandering PCB strips 802_{1,2} connected at the long sides of the elongated rigid PCB move downwardly wherein the meandering part of the flexible PCB strips enable the flexible PCB strips to exit the hollow cushion-shaped protruding element at its base. When mounted in the protruding element, the meandering shape of the flexible PCB strips and the two-sided connection of the flexible PCB strips will avoid or at least substantially reduce interferes of the electronic connection of the sensor module with the mechanical characteristics, i.e. the deflective and/or compressive characteristics, of the protruding element. When mounted into the sensor head, the thin flexible PCB strips allow the sensor module to rotate about various axis, in particular the z-axis and the y-axis or combinations thereof, and allow translation of the sensor module in the direction perpendicular to its plane (the z-y plane in FIG. 7). This way, the protruding element comprising the electrically connected sensor module can be deflected and/or compressed in different (rotational and translational) directions as indicated in FIG. 8.

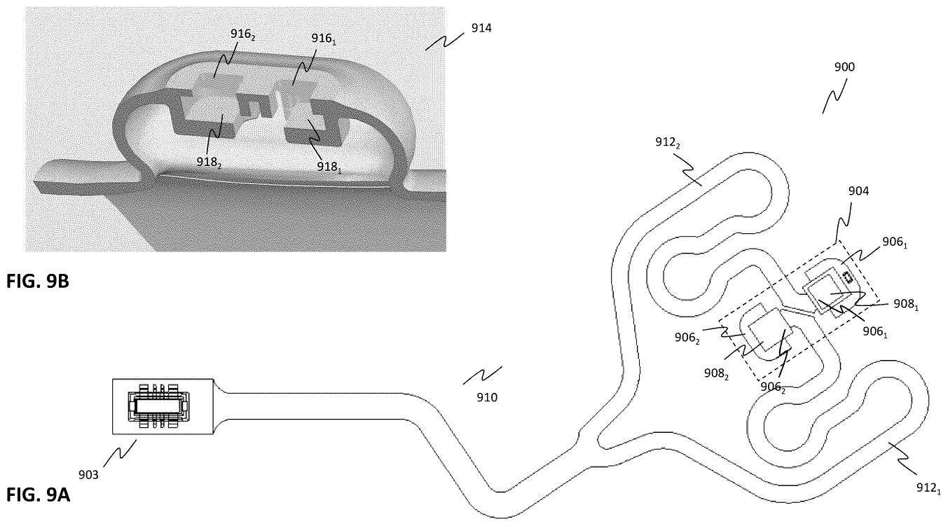

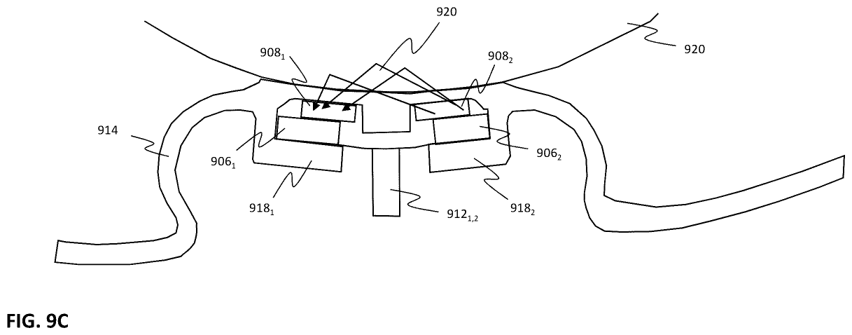

FIG. 9A-9C illustrate a flexible printed circuit board structure connected to a sensor module and a flexible protruding element for a sensor assembly according to another embodiment of the invention. As shown in FIG. 9A, the flexible printed circuit board structure 902 may be connected to a sensor module 904 and electronics 903, e.g. an analog stage for processing signals originating from the sensor module. The flexible printed circuit board structure may include two narrow PCT strips 912_{1,2} merging into one PCB strip 910 connected to the electronics 903. In this embodiment however, the sensor module may include two separate rigid or semi-rigid PCBs, each having one or more electrical elements mounted thereon. Hence, in this case, the sensor module may include a light emitting element 908₁ mounted on a first PCB 906₁ and a light sensor element 908₂ mounted on a second PCB 906₂.

The first and second PCB may be inserted into a flexible protruding element as shown in FIG. 9B. As the sensor module include two separate PCBs connected to the two narrow PCB strips, the hollow protruding element may include two recess structures, a first recess 918₁ and a second recess 918₂, in which the PCBs forming the sensor modules may be fitted. The recesses may be shaped so that the first PCB 906₁ comprising the light emitting element 908₁ and the second PCB 906₂ comprising the light sensor element 908₂ are fixated and pressed against the inner side of a first and second transparent windows 916_{1,2} respectively. Using two separate PCBs for mounting different elements of the sensor module, e.g. one or more light emitting devices and one or more light sensing devices, allows both PCBs to move independently from each other.

FIG. 9C depicts a cross-section of a flexible protruding element, housing a sensor module according to an embodiment of the invention. As shown in this figure, the flexible protruding element is pressed against a curved or irregular part of the skin so that the two sensor elements (forming a reflective type sensor such as a reflective SpO₂ sensor) have a different orientation with respect to each other due to the fact that the sensor elements are independently mounted in the flexible protruding element. When the flexible protruding element is pressed against the irregular/curved surface of the skin, the protruding element will deform so that the surface of the contact area follows the curvature of the surface of the skin. This way, the emitting surface of the LED and the light receiving surface of the photosensor is faced towards the surface of the skin. Further, the sensor elements are electrically connect via a flexible or stretchable PCB strips to the electronics. As shown in this figure, despite the irregular surface of the skin, the sensor elements will always be close to the skin without any interfering airgap between the skin and the contact area. This way light 920 emitted by the light emitting element will efficiently penetrate the skin and scattered/reflected by the tissue. The scattered/reflected light that travelled through the tissue is subsequently detected by the light detecting element.

The reason why the sensor elements need to be in continuous contact with the skin for an optimal sensor signal can be understood by the fact that when computing the oxygen saturation (SpO₂) the so-called Beer-Lambert Law is used to relate the incident intensity (detected by a photosensor) to the transmitted intensity (transmitted by the light emitting diode). For a detailed description of the calculation reference is made to the handbook by Webster J G. Design of Pulse Oximeters. Bristol: Institute of Physics Publishing, 1997.

In case of a biological homogenous medium the model may be described by the following equation

Iₜ=Iᵢe^{-B-ϵαl}

wherein B is a background term characterizing all of the tissue besides blood, α is the extinction coefficient in blood and l is the effective path length. This model may be rewritten to a measurable ratio R which can be used to compute the oxygen saturation in blood based on two wavelengths:

$${SO}_{2} = \frac{{ϵ}_{d 1} - R {ϵ}_{d 2}}{R ( {ϵ}_{o 2} - {ϵ}_{d 2} ) - ( {ϵ}_{o 1} - {ϵ}_{d 1} )} .$$

Herein, index o denotes oxyhemoglobin, index d denotes deoxyhemoglobin, indices 1 and 2 denote the first and second wavelength, respectively, and R denotes the ratio of ratios. One assumption in this model is that the optical pathlengths between the two wavelengths are substantially identical so that this term can be eliminated. This assumption implies that the scattering behaviour of both wavelengths is also identical. For measuring SpO₂, a red led of 660 nm and an infrared led of 940 nm is often used. The scatting properties of both wavelengths are not identical so that a one-off calibration is needed.

The above-described approach is based on ideal circumstances such as homogeneous biological tissue and the sensor having continuous contact with the skin. In real situations this is not the case. Each ear has a different geometry (differently curved and/or different irregulars skin surface) which may introduce space the LEDs and/or detector and the skin, causing scattering of light. This way, the detector may detect scattered light that has not travelled through the skin so that the measured sensor signal is not reliable any more.

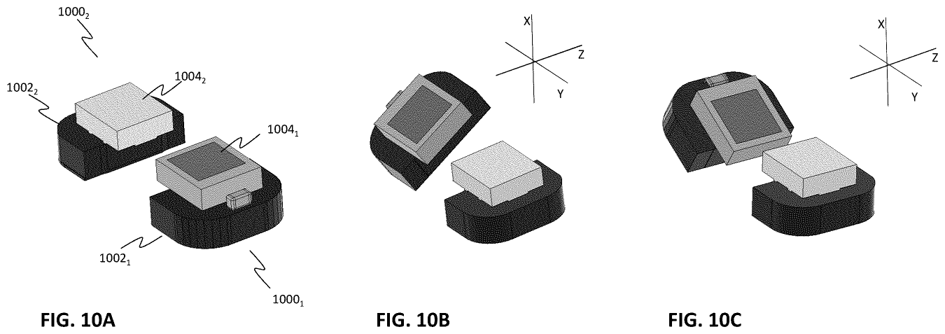

Mounting the sensor elements in the flexible sensor head as described with reference to FIGS. 9 and 10 solves this problem. The sensor elements can be orientated independently form each other so that the sensor elements are always in contact with the skin. Although the absolute path length may differ per person, the relative ratio between the pathlength of the two different wavelengths will be the same so that the path lengths can be removed from the computation. This way, only one calibration curve is needed. A further advantage is that the sensor elements, the LEDs and the photosensor may be spaced further apart as they will follow the curved surface of the skin. This will increase the perfusion underneath the sensor and hence increase the quality and reliability of the sensor signal.

FIG. 10A-10C schematically show two sensor elements 1000_{1,2} can be oriented in the flexible protruding element when the contact surface is pressed against the skin. Each sensor element may include a PCB part 1002_{1,2} and a sensor part 1004_{1,2}. FIG. 10A shows the basic orientation of the two sensor elements in a flexible protruding element when it is pressed against a substantially flat surface. In that case, the surface of the sensor elements may be substantially similar. In contrast, non flat surfaces may cause changes in the relative orientation of the two sensor elements with respect to each other. For example, FIG. 10B illustrates a situation wherein the sensor elements are slightly rotated with respect to each other around a longitudinal axis of the sensor module. Hence, in this case, the orientation of the surface of the first sensor element differs from the orientation of the second sensor element. Similarly, FIG. 10C illustrates a situation wherein the two sensor elements are lightly rotated with respect to each other around an axis perpendicular to the longitudinal axis. The different orientations of the sensor elements (“twist” and “bend”) may be realized due to the flexibility of the protruding element. This way, when the contact surface of the flexible protruding element is pressed against an irregular skin surface, the sensor elements may follow the irregular surface easier. Thus, despite the irregular surface, the chance of forming an air gap between a sensor element and the skin which causes degraded sensor signals is reduced and the signal quality of the senor module may be improved.

Thus, the sensor assembly may include a fixation means to keep a hollow flexibly deformable protruding element (a flexible sensor head) in contact with skin of a wearer of the sensor assembly. The structure of the protruding element will have a predetermined cushion-shape with a contact area which may be formed as an integral part using (injection) molding techniques. The protruding element includes a base and flexible side walls, e.g. a narrow concave shaped based and convex shaped sidewalls that connect the base with the contact area. This way, the protruding element will flexibly (reversibly) deform in a controlled way if a force is applied to the sensor assembly. In particular, the protruding element will deform so that the contact area will increase when the applied force will increase. Further, the interior of the flexible sensor head may include a fixating structure for fixating and pressing sensor elements, e.g. light emitting and light sensing devices, against the inner surface of a transparent window in the contact area of the flexible sensor head.

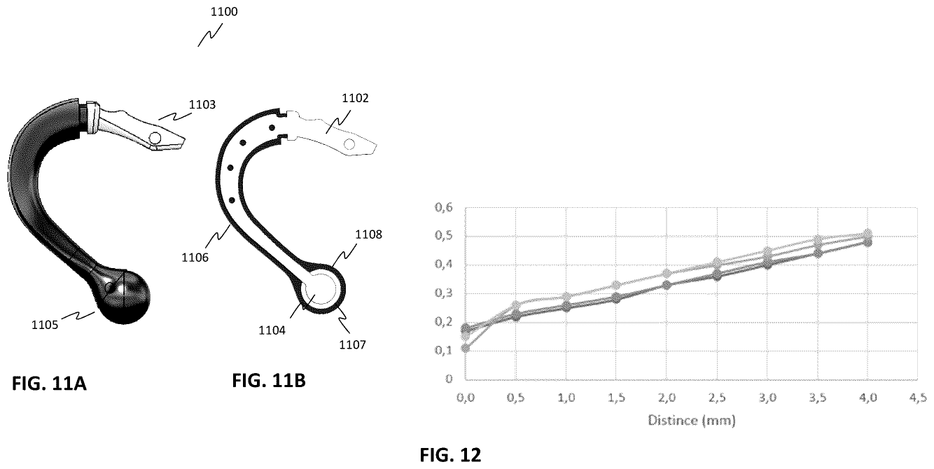

FIGS. 11A and 11B illustrate an example of ear hook 1100 for an ear worn sensor device according to an embodiment of the invention. FIG. 11A depicts a side view of the hook which may be shaped to fit the backside of an ear. A first end of the hook may include a spherical element 1105 forming a balloon-shaped contact surface for engaging with the inner side of the ear. A second end of the arm may include a connecting structure 1103 configured to engage with a connecting structure of the electronics housing (as e.g. shown in FIG. 1A). FIG. 11B depicts a cross-section of the elastomeric arm show that the arm includes an elastomeric arm 1102 and a flexible, silicone cover 1106 provided over the elastomeric arm to provide an outer cover for comfortable wear. In an embodiment, the thickness of the sheet material forming the cover may be selected between 0.5 and 2 mm. Further, as shown in FIG. 11B, the spherical contact structure may include a spherical element 1104 that is connected (or being part of) the elastomeric arm 1102. Further, the flexible silicone cover 1106 may be arranged over the spherical element to form the balloon-shaped contact structure. The radius of the spherical element is smaller than the radius of the balloon thus defining an airtight space 1107 between the spherical element. In an embodiment, dimensions (radius) of the spherical element may be between 3 and 12 mm. In a further embodiment, dimensions (radius) of the balloon-shaped flexible silicon cover may be tween 6 and 14 mm. Further, the height of the space between the spherical element and the balloon may between 1 and 4 mm.

The airtight space may be filled with air or a gas wherein when the sensor is not in use the pressure within the airtight space is approximately equal to the pressure outside the space. This way, the silicone balloon will have a compressible sphere-shaped contact surface. This way, when the ear sensor is worn around, the contact pressure between the balloon-shaped contact structure can be kept below 7 kPa and, preferably at an (average) contact pressure between 2-3 kPa on average. This way, skin irritation or even skin damage due to as long-term exposure to a force against skin can be avoided.

In use, the sphere-shaped contact surface of the ear hook presses against inner side of the ear, while the contact surface of the protrusion presses against the back side of the ear. The force exerted on the inner and back side of the ear will be defined by the opening between the two contact surfaces and the spring constant of the structure. When used, typically the distance between the two contact surfaces will be between 1-5 mm. As shown in FIG. 12, such opening distances are associated with forces in the range of 0.2-0.6 N. For the contact pressure, the contact area is necessary, which is difficult to determine in an accurate way as the contact surfaces will deform depending on the shape and dimension of the ear. For a contact surface of about 28 mm² (3.5 mm×8 mm) an average contact pressure will be in the range between 9 and 14 kPa. It is noted that these values may depend on the geometry and material properties of the sensor head, the hook and the spherical element. When worn, these pressures may be lower as the contact surface will increase when the sensor head is pressed the backside of the ear.

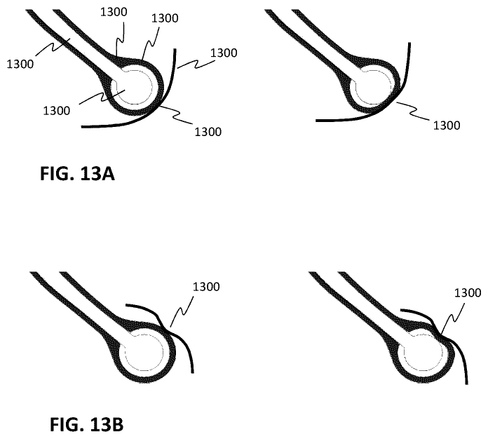

FIGS. 13A and 13B illustrate the elastic properties of the balloon-shaped contact structure of the ear hook according to an embodiment of the invention. As shown in these figures, a part of an ear hook 1300 of an elastic material similar to the one described with reference to FIGS. 11A and 11B, may comprising a spherical contact structure for contacting an inner part of the ear 1302. The contact structure may include a spherical element 1304 connected to the hook and a balloon-structure 1304 of a flexible sheet material 1306 formed around the spherical element, wherein the radius of the balloon structure is larger than the radius of the spherical element. This way a air gap is formed between the solid spherical element. The space between the spherical element and the balloon structure may airtight. This way, the compressible gas, typically air, may contribute to the spring/elastic properties of the spherical contact structure. The width of the airgap between the outer surface of the spherical element and the inner surface of the balloon-structure being selected between 0.5 and 4 mm, preferably between 1 and 3 mm.

As shown in FIG. 13A the spherical contact structure is shaped to contact the curved inner side of the ear. Thus, when the ear hook is attached to the ear, the elastic properties of the spherical contact structure causes compression of the balloon structure up to a point that the sheet material of the balloon structure touches the spherical element. Hence, the width of the air gap will determine to which extend the spherical contact structure can be compressed. FIG. 13B illustrates that the spherical contact structure can cope with difference surface irregularities, such as local protrusions in the skin. Hence, the compressive/elastic properties of the spherical contact structure is particularly suitable for contacting an inner part of an ear and to apply a pressure which is necessary to fixate the air worn sensor device as described with reference to the embodiments described in this application.

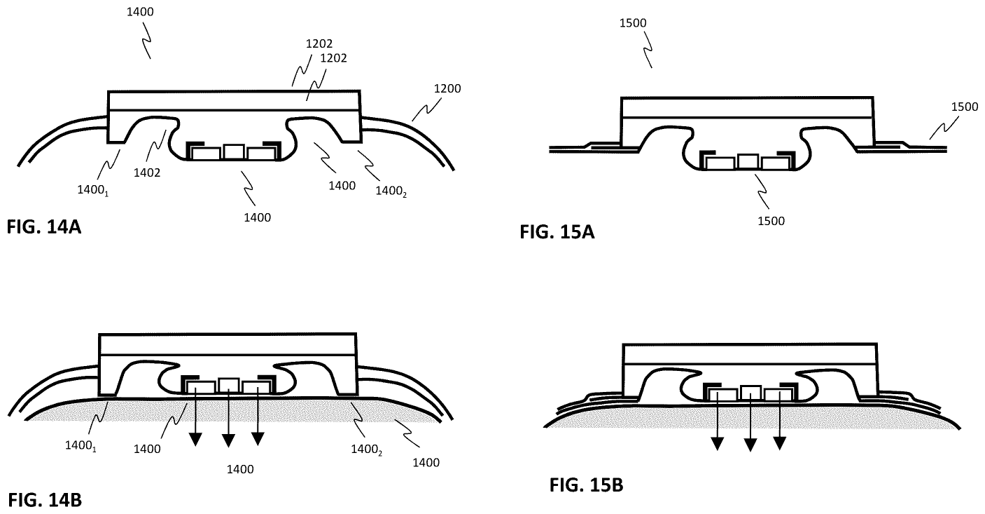

It is submitted that while the sensor assemblies according to the embodiments in this application are described with reference to an ear-worn sensor assembly. Such implementation should be regarded as a non-limiting implementation. Other types of sensor assemblies, e.g. a sensor assembly attached to a finger or attached to the skin based on a plaster are also foreseen without departing from the essence of the invention. Examples of such embodiments are shown in FIGS. 14 and 15, which show cross-sectional figures of sensor devices implemented as a wrist worn sensor or as a plaster or a patch.

For example, FIG. 14A depicts a cross-sectional view of a wrist-worn sensor device 1400 including a protective casing 1402 comprising a top surface and side walls 1404_{1,2}. The casing may include a compartment for housing electronics 1406. Further, the bottom side of the protective casing may include a peripheral part 1408_{1,2} and a flexible sensor head 1410, a flexibly deformable protruding element, connected to the peripheral part. The peripheral part may include a surface for positioning the sensor device on a part of the wrist. The flexible sensor head may include a contact surface for contacting skin of a wearer. The dimensions of the flexible sensor head are selected such that the contact surface extends beyond the surface of the peripheral surface, so that when the sensor device is attached to the wrist, the sensor head will be compressed. The compression will depend on a height difference 1403 defined by the difference of the height of the contact service and the height of the surface of the peripheral part. The sensor head may house a sensor module which is fixated against the inner side of contact surface in a similar way as depicted in FIG. 3. The sensor module may be fixated against the inner surface of the contact surface according to any of the embodiments described in this application. The deformable protruding element may be hollow, defining a chamber or a cavity, comprising flexible side walls 1412 connecting a contact surface 1414₁ (the apex) via a base 1416 to the peripheral part of the protective case. The contact surface and the one or more side walls may be made out of a flexible material such as a silicone or a rubber material that has a high friction coefficient with dry skin. Wrist bands 1418 may be connected to the protected casing so that the sensor device may be attached to the wrist.

FIG. 14B depicts the sensor attached to the wrist. As shown in the figure, when attached to the wrist, the peripheral part 1408_{1,2} of the protective casing will rest onto the skin of the wearer. As the contact surface of the sensor heads extends beyond the surface of the peripheral part, contact surface of the sensor head will be pressed against the skin with a certain pressure 1422 which depends on the spring constant of the flexibly deformable protruding element. The elastomeric properties and the dimensions of the protruding element may be designed such that the applied pressure is within a range allowing long wear of the sensor in combination with an optimal sensor signal due to the fact that the contact surface of the protruding element will be fixated against the skin under all circumstances. FIGS. 15A and 15B depict a sensor device 1500 that is configured as a plaster or a patch. The sensor device may include a protective casing and a flexible sensor head comprising a sensor module that is similar to the one described with reference to FIG. 14A. In this case however, an attachment structure 1502 connected to the peripheral part may include a surface 1504 comprising adhesive to attach the sensor device as a plaster to a body part.

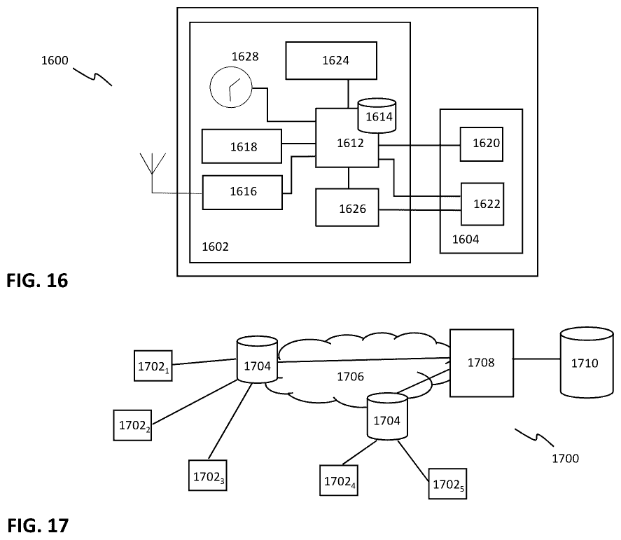

FIG. 16 depicts a schematic of functional components a wearable sensor assembly according to the embodiments in this application could comprise. As shown in the figure, the wearable sensor assembly 1600 may comprise a digital electronics module 1602, a sensor module 1604. The digital electronics module may comprise a microprocessor 1612 coupled to a memory 1614, a rechargeable battery 1624, a radio interface 1616 (based on a suitable wireless protocol such Bluetooth, ZigBee, Wi-Fi, ANT, etc.) and/or a wired interface, e.g. USB, etc. The digital electronics module may also include one or more sensors, e.g. a motion sensor 1618, e.g. a digital accelerometer for monitoring motion. Further, the sensor chip may comprise and one or more sensors for sensing vital sign parameters. For reliable determination of these parameters the sensors need to be close to the body (skin) of the person wearing the sensor assembly.

Non-limiting examples of such sensors include (but are not limited to), a temperature sensor 1620 and at least one opto-electronic sensor 1622. The output of the one or more sensors may be fed into the input of an analog circuit module 1626, which may comprise a low noise pre-amplifier and filtering, so that sensor signals can be amplified before they are processed by the microprocessor. Typically, for optimal use of the analog circuit block, the pre-amplifier and filtering needs to be located close to the sensor chip. Therefore, in some embodiments, both the sensor chip and the analog circuit block may be located in the flexible 3D sensor housing, while the digital electronics card may be located further away from the sensor chip

At regular intervals, the sensors may be activated based on a motion signal of the motion detector, sensor signals may be accepted or rejected on the basis of a motion signal and accepted sensor signals may be processed in order to determine vital sign parameters such as SpO₂ level, hear beat rate, temperature and motion associated with a particular time instance. The determined vital sign parameters may be time stamped on the basis of a clock 1628, stored in the memory 1614 and transmitted at regular intervals to a base station.

FIG. 17 depict schematics a low-care vital sign monitoring system 1700 comprising a plurality of wireless vital sign measuring devices 1702₁₋₅ which may be implement in accordance with the embodiments described in this application. As already described with reference to FIG. 16, the vital sign measuring devices may be configured to wirelessly communicate with one or more base stations 1704_{1,2}. A base station may be configured to receive data from different measurement devices that are located within a certain distance from the base station.

A measuring device may include one or more sensors that are in contact with or in close vicinity to the skin of a person wearing the measuring device so that the sensors can measure characteristics, e.g. an optical response and/or temperature, of the skin tissue. Additionally, one or more sensors, e.g. an accelerometer, may be configured to generate motion information associated with the movements of the person wearing the measuring device and/or posture information associated with the posture of the person wearing the measuring device.

At predetermined (measuring) time instances, the measuring device may determine on the basis of the measured sensor signals one or more vital sign parameters, e.g. heartbeat, oxygen saturation (SpO₂), temperature, posture, etc. of the person wearing the measuring device. The measuring time instances may be periodically (every N minutes or every N hours or the like), a-periodically (e.g. depending on certain conditions) or a combination thereof.

The measurement device may process signals generated by the sensors (sensor signals) on the basis of motion data that are measured during the measuring the data that are used for determining the vital sign parameters. The thus processed sensor signals may be used to calculate one or more vital sign parameters. The vital signal parameters may be temporarily stored before transmitting the parameters in one or more messages via a wireless interface, e.g. a radio interface, to a base station. A central computer 1708 may be connected via one or more networks 1706 to the base stations of the vital sign measuring system may receive the vital sign parameters and monitor the measured parameters of different patients in time.

In other variants, the measuring device may determine and store sensor signals and transmit the sensor signals via a base station to the central computer, which may include a processor for calculating the one or more vital sign parameters based on the sensor signals.

The monitoring process may include determining a trend of one or more vital sign parameters and triggering a warning signal in case at least one of the vital sign parameters (or the trend of at least of the vital sign parameters) indicates a (substantial) deterioration of at least one of the vital sign parameters (or trend therein).

At least part of the vital sign parameters may be derived from pulse oximetry, which is a non-invasive method for monitoring a person's oxygen saturation. A blood-oxygen saturation reading indicates the percentage of hemoglobin molecules in the arterial blood which are saturated with oxygen. The term SpO₂ means the SaO₂ measurement determined by pulse oximetry. Other vital sign parameters may be derived from blood perfusion levels in the tissue based on so-called photoplethysmography (PPG) measurements.

The vital sign sensor may include opto-electronic sensor including one or more light emitting devices, e.g. LEDs, adapted to emit light of a predetermined wavelength or a predetermined band of the electro-magnetic spectrum onto a tissue and one or more light sensors, e.g. photodiodes, adapted to receive LED light that is reflected from the tissue or transmitted through tissue. In particular, light emitting diodes may expose part of the tissue to red and infrared light and one or more light detectors, e.g. a photodiode, may.

The amount of light received by the detector provides an indication of the amount of oxygen bound to the hemoglobin in the blood. Oxygenated hemoglobin (oxyhemoglobin or HbO₂) absorbs more infrared light than red light and deoxygenated hemoglobin (Hb) absorbs more red light than infrared light. Thus, by detecting the amount of red and infrared light transmitted through or reflected from the tissue an SpO₂ value may be determined. An SpO₂ sensor may be attached to a body part having relatively translucent skin tissues, typically to an extremity of a body part such as a finger, toe or ear. Further, in contrast to high-care applications, in low-care applications, a user is not bound to bed but should be able to walk around and move freely. Hence, for that reason, the measuring device may be configured as a small wireless wearable device that is in contact with a part of the skin. As will be described hereunder in more detail, in an embodiment, the measuring device may be configured as an ear-worn wireless measuring device wherein the measuring device comprises an opto-electronic sensing part that is in contact with the skin of (the back of) an ear of the person wearing the measuring device.

In order to control the power consumption, the measurement device can switch between an idle state and an active state. In the idle state, the opto-electronic sensor is in a low-power (sleeping) state, wherein the opto-electronic sensor is deactivated. In contrast, the low-power motion sensor is still active so that it can generate motion information that can be used by the processor to switch the measuring device to the active state in which the opto-electronic sensor is activated so that during a predetermined period of time optical data can be measured. Once the measurement period is over, the processor may switch back to the idle state. Hence, the low-power motion sensor may continuously or at least regularly or periodically measure the motion of the patient wearing the measuring device wherein the motion information may be used to switch the measuring device from an idle state to an active state, wherein the opto-electronic sensor is activated and wherein the processor may decide to activate the wireless interface in order to transmit at least part of the measured data to a base station.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A wearable sensor device for contacting skin comprising:
a hollow, flexibly deformable protrusion comprising a base and an apex, at least part of the apex forming a contact area for contacting the skin;
a sensor module mounted on or inside the protrusion, the sensor module being mounted on or against the contact area; and,
a sensor device fixating structure configured to press the contact area with a substantially constant pressure against the skin, wherein, when the contact area is pressed against the skin, an increase, respectively decrease of a force exerted on the sensor device deforms, e.g. deflects and/or compresses, the protrusion such that the contact area that is in contact with the skin increases, respectively decreases, while the pressure stays substantially the same.

2. The wearable sensor device according to claim 1, wherein the pressure is smaller than 8 kPa.

3. The wearable sensor device according to claim 1 wherein the base is configured as a flexible joint region connecting the protruding element to the fastening structure, the flexible joint region providing flexible movement of the protrusion in rotational, translational and/or angular directions relative to the base part.

4. The wearable sensor device according to claim 1 wherein at least part of the sensor module is fixated inside the protrusion against a sheet material forming the contact area of the protrusion.

5. The wearable sensor device according to claim 1 wherein the hollow flexibly deformable protrusion is made of an elastomeric sheet material.

6. The wearable sensor device according to claim 1, wherein at least part of the contact area is made of a flexible opaque material, the contact area further comprising one or more transparent windows of a flexible optically transparent material, the sensor module being an optical sensor module comprising one or more optical sensor elements, the optical sensor module being mounted against the backside side of the contact area in alignment with the one or more transparent windows.

7. The wearable sensor device according to claim 6 wherein surface the contact area comprises one or more recesses at the position of the one or more transparent windows, the one or more recesses being configured to house at least part of the one or more optical sensor elements respectively.

8. The wearable sensor device according to claim 1 wherein the sensor module comprises at least a light detecting element and a light emitting element, optionally a light blocking member being positioned between light detecting and the light emitting element for optically isolating the light detecting element from light that arrives at the light detecting element other than via one of the optical windows.

9. The wearable sensor device according to claim 1, wherein the protruding element comprises a cavity or a chamber formed by the apex and one or more side walls connecting the apex to the base.

10. The wearable sensor device according to claim 9 wherein at least part of the one or more elastic walls have a convex shape so that when the protruding element is deflected and/or compressed, the one or more side walls will deflect outwardly.

11. The wearable sensor device according to claim 9 wherein the cross-sectional dimensions of the protruding element at the joint region are smaller than the cross-sectional dimensions of the protruding element at a height of the side walls.

12. The wearable sensor device according to claim 1 wherein the sensor device further comprises a housing comprising electronics for processing sensor signals of the sensor module, one or more strips of a flexible and/or stretchable PCB electrically connecting the sensor module to the electronics.

13. The wearable sensor device according to claim 1 wherein the sensor module includes a first sensor element mounted against a first part of an inner side of the material forming contact area and a second sensor element mounted against a second part of the material forming the inner side of the contact area so that, when the outer surface of the contact area is pressed against the skin, the first sensor element can have an orientation that is different from the orientation of the second sensor element.

14. The wearable sensor device according to claim 1 wherein the sensor module comprises an elongated rigid PCB board having a longitudinal axis and a transversal axis, the rigid PCB comprising two or more sensors elements mounted thereon and at least two flexible PCB strips, each flexible PCB strip being connected to a long side of the rigid PCB board so that the rigid PCB board can rotate about its longitudinal axis and its transversal axis.

15. The wearable sensor device according to claim 1 wherein the sensor device fixating structure is configured to fixate the contact area against the skin of the backside of the ear.

16. The wearable sensor device according to claim 1 the chamber or cavity of the protruding element is filled with a compressible material.

17. The wearable sensor device according to claim 1 wherein the wearable sensor device is configured as an ear sensor, the sensor device fixating structure including an ear hook of an elastic material configured to press the contact area against skin of the backside of the ear, a first end of the ear hook being sphere shaped contact structure, the sphere shaped contact structure a spherical element and a balloon-structure of a flexible sheet material formed around the spherical element.

18. The wearable sensor device according to claim 1 wherein the wearable sensor device is configured to be attached to a body part, e.g. as a wrist-worn sensor or a plaster, further comprising:
a protective casing comprising a top surface, side walls and a bottom surface; and
the bottom surface including a peripheral part, the flexibly deformable protruding element being flexibly connected to the peripheral part, the peripheral part including a surface for positioning the sensor device against the skin;
wherein the sensor device fixating structure is configured to fixate the peripheral part against the skin and/or wherein the contact area of the protruding element extends beyond the surface of the peripheral part, so that when the sensor device is fixated against the skin, the contact area will be pressed against the skin with a predetermined pressure.

19. The wearable sensor device according to claim 1, wherein the protrusion is a cushion or a balloon-shaped protrusion and/or wherein the apex is a substantially flat apex.

20. The wearable sensor device according to claim 1, wherein the module fixating structure includes a flexible clamping structure for clamping the sensor module around the ear so that the contact area is pressed against the skin of the backside of the ear.
